(19) United States
(12) Patent Application Publication
(10) Pub. No.: US 2018/0294049 A1
(43) Pub. Date: Oct. 11, 2018

(54) OPIATE REDUCTION TREATMENT SYSTEM

(71) Applicant: ROCA MEDICAL LTD., London (GB)

(72) Inventors: James STRADER, Austin, TX (US); Jovan Hutton PULITZER, Frisco, TX (US); Edmund Dennis HARRIS, Lakehills, TX (US)

(21) Appl. No.: 15/946,413

(22) Filed: Apr. 5, 2018

Related U.S. Application Data
(60) Provisional application No. 62/482,040, filed on Apr. 5, 2017.

(51) Int. Cl. G16H 20/10; G16H 50/30; G16H 50/70

(52) U.S. Cl. CPC G16H 20/10; G16H 80/00; G16H 50/70; G16H 50/30

(57) ABSTRACT

This disclosure relates to an opiate reduction treatment system. The system comprises a PIN generator for creating a Patient Identification Number (PIN) unique to a given patient, wherein the PIN includes one or more fields, and wherein the one or more fields each include a scored value, each scored value associated with a defined portion of a health profile of the given patient, a database including test results for a plurality of PINs, and known treatments, and a neural network, including an input layer configured to receive an output of a PIN for a given patient from the PIN generator and compound constituents as input values, an output layer configured to provide an opioid reduction treatment prediction, an intermediate layer configured to store a representation of the database, and map the input layer to the output layer through the stored representation.

### CROSS-REFERENCE TO RELATED APPLICATIONS

This application claims the benefit of U.S. Provisional Application No. 62/482,040, filed on Apr. 5, 2017, entitled OPIATE REDUCTION TREATMENT SYSTEM (Atty. Dkt. No. RCMD-33519) which is incorporated by reference herein in its entirety.

### TECHNICAL FIELD

The following disclosure relates to opioid abuse and systems and methods for reduction of the use of opioids.

### BACKGROUND

Opioids are medications that treat pain in many contexts, from post-surgical relief to chronic severe back pain and of-like care. Two of the most common forms are oxycodones, often sold under the brand names OxyContin® and Percocet®, and hydrocodones, sold as Vicodin®. Both are powerful narcotics. Americans are the number one consumer of these drugs, accounting for almost 100 percent of the hydrocodone prescriptions and 81 percent of oxycodone prescriptions worldwide. In the United States, more than 2 million people are addicted to these medications.

These drugs became more readily available to patients in the late 1990s, and prescription rates nearly doubled between 1998 and 2013. This epidemic is the unintended consequence of policy and practice that was supposed to benefit patients and keep them safe. A solution to this kind of systemic problem that affects the health, social, and economic welfare of society requires a large-scale, comprehensive course of action. The healthcare delivery system is ground zero.

The result in recent years is opioid overuse and over prescription. However, pain relief is critically important to a number of patients and the use of opioids in relieving this pain is the primary avenue chosen by most physicians. The problem facing healthcare industry is: too little pain relief and millions will suffer; too much and lives are at risk. The challenge facing the healthcare industry is to solve this problem and, at the same time, realize a significant reduction in opioid use.

### SUMMARY

In one aspect thereof, an opiate reduction treatment system is provided. The system includes a PIN generator for creating a Patient Identification Number (PIN) unique to a given patient, wherein the PIN includes one or more fields, and wherein the one or more fields each include a scored value converted from raw data corresponding to one or more test results, each scored value associated with a defined portion of a health profile of the given patient, a database including test results for a plurality of PINs for a plurality of patients and associated compound constituents provided to each of the plurality of patients, and known treatments, and a neural network, including an input layer configured to receive an output of a PIN for a given patient from the PIN generator and compound constituents as input values, an output layer configured to provide an opioid reduction treatment prediction, an intermediate layer configured to store a representation of the database, and map the input layer to the output layer through the stored representation.

In one embodiment, the scored value is created from one or more inputs from the raw data that are weighted according to associated test types and normalized.

In one embodiment, the one or more fields of the PIN includes a code assigned to a patient.

In one embodiment, the code assigned to the patient is a Patient Information Profile (PIP), wherein the PIP identifies the patient.

In one embodiment, at least one of the one or more fields of the PIN corresponds to a particular test.

In one embodiment, at least one of the one or more fields of the PIN corresponds to a compound formulation.

In one embodiment, the scored value is a value within a number range.

In one embodiment, the number range is a range between 1 and 10.

In one embodiment, the PIN represents a patient pain profile at a first point in time.

In one embodiment, the neural network is further configured to receive an output of another PIN representing a patient pain profile at a second point in time, predict another opioid reduction treatment using the other PIN, and store a revised treatment plan in the database.

In another aspect thereof, a method for providing an opiate reduction treatment is provided. The method includes generating a Patient Identification Number (PIN) including one or more fields, collecting raw data corresponding to one or more test results, converting the raw data into a scored value, storing the scored value in one of the one or more fields of the PIN, predicting an opioid reduction treatment for a patient, including providing as input values an output of the PIN and compound constituents to an input layer of a neural network, applying, by an intermediate layer of the neural network, the input values and compound constituents information to a stored representation of a database, wherein the database includes test results for a plurality of PINs for a plurality of patients and associated compound constituents provided to each of the plurality of patients, and generating, by an output layer of the neural network, an opioid reduction treatment prediction, and delivering to a patient an opioid reduction treatment corresponding to the opioid reduction treatment prediction.

In one embodiment, converting the raw data into the scored value includes creating one or more inputs from the raw data, applying a weight to the one or more inputs to generate one or more weighted results, each one of the one or more weighted results corresponding to one of the one or more inputs, summing the one or more weighted results to generate a summed output, dividing the summed output by a number of tests to generate a result, and translating the result into the scored value.

In one embodiment, the one or more fields of the PIN includes a code assigned to a patient.

In one embodiment, the code assigned to the patient is a Patient Information Profile (PIP), wherein the PIP identifies the patient.

In one embodiment, at least one of the one or more fields of the PIN corresponds to a particular test.

In one embodiment, at least one of the one or more fields of the PIN corresponds to a compound formulation.

In one embodiment, the scored value is a value within a number range.

In one embodiment, the number range is a range between 1 and 10.

In one embodiment, the PIN represents a patient pain profile at a first point in time.

In one embodiment, the method further includes providing an output of another PIN representing a patient pain profile at a second point in time, predicting another opioid reduction treatment using the other PIN, and storing a revised treatment plan in the database.

### BRIEF DESCRIPTION OF THE DRAWINGS

For a more complete understanding, reference is now made to the following description taken in conjunction with the accompanying Drawings in which:

FIG. 1 illustrates a flowchart for the initial patient visit;

FIG. 2 illustrates a diagrammatic view of the overall process for creating a Pain Centric Patient PIN;

FIG. 3 illustrates a histogram for creating binned values for populating the Pain Centric Patient PIN;

FIG. 4 illustrates a flowchart for the Bin process;

FIG. 5 illustrates a flowchart for the consolidation operation;

FIG. 6 illustrates a diagrammatic view of one set of test results that are used to generate a value for the binning operation;

FIG. 7 illustrates a diagrammatic view for the consolidation operation to normalize multiple tests into a score;

FIG. 8 illustrates the operation wherein the PIN is mapped through a model of the compounding process;

FIG. 9 illustrates a diagrammatic view of a nonlinear network for realizing the overall model; and

FIG. 10 illustrates a schematic view of a neural network.

### DETAILED DESCRIPTION

In order to reduce opioid use, other compounds are resorted to. These involve, in some cases, topical analgesics which are used to reduce systemic exposure to opioids, limit side effects, and lower the risk of drug-drug interactions. The goal of utilizing these alternative or other compounds is to improve tolerability and reduce overall opioid use—all while managing primary pain symptoms. However, most people with chronic pain have a desire to do anything possible to get rid of the pain. Their first introduction to any pain medication in the healthcare system will be through their primary physician and, even though they may come to the physician asking for a particular medication by name or simply asking for the strongest drug they are offering, the healthcare system has a desire to reduce the influence of pain as opposed to getting rid of the pain, through such things as providing patients with realistic expectations and teaching acceptance of pain itself. However, pain medications in the form of opioids will still be a mainline treatment.

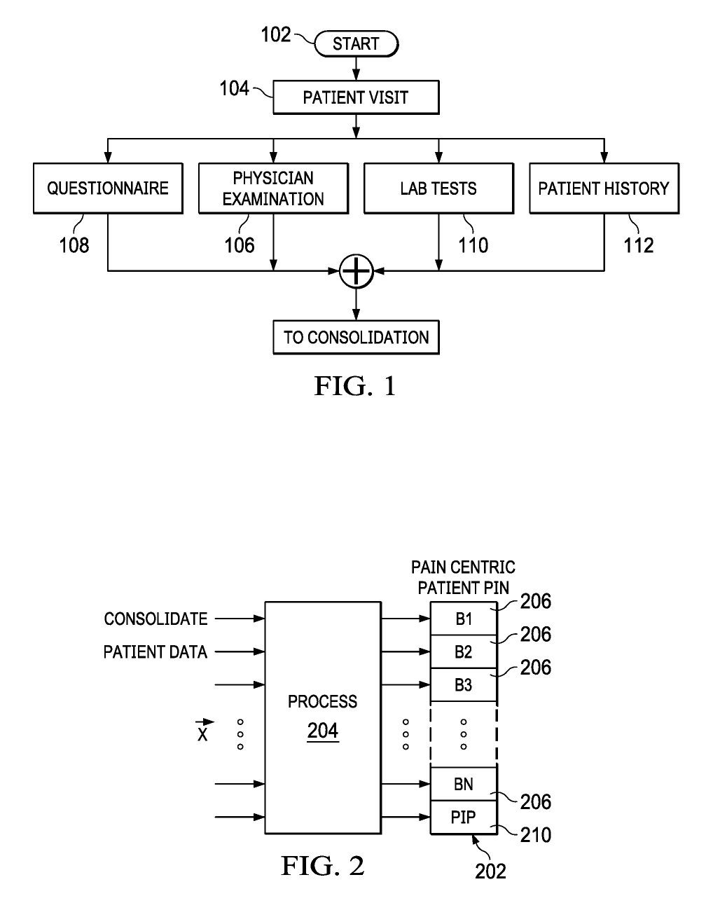

Referring now to FIG. 1, there is illustrated a diagrammatic view of the first step in determining what compound possibly might be useful to achieve opioid reduction. The primary interface to the medical system will be the primary physician. The primary physician can evaluate a particular patient through a physical exam, evaluating drug tests that are specifically focused on drug use and pain, keeping in mind that each patient is unique in their source of the pain and in their therapeutic regimen that they may follow. In addition, this can change over time as a result of using opioids, understanding that chronic pain is very closely tied with the interplay of various physical limitations, psychosocial sequelae, personality predispositions, stress, medical uncertainty, and personal coping resources.

Initially, the process is initiated at a block 102 and proceeds to a block 104 which represents the overall patient visit, the first interface of the patient to the healthcare system. In this patient visit, and specifically one with the purpose of reducing opioid use, it is recognized that the patient uses some form of opioid at some level. The physician at this point utilizes a physical examination of block 106, a questionnaire at block 108, lab tests at block 110, and patient history at block 112 in order to collect data on a particular patient at a particular time. This will allow a profile of the patient to be determined. And this profile will be altered somewhat by the results of some of the lab tests and some of the results of the physical examination. This examination may be physical, and it may be psychiatric in order to address various comorbid states, such as depression, anxiety, and post-traumatic stress disorder. Chronic pain and depression, in particular, are intense bedfellows.

Referring now to FIG. 2, there is illustrated a diagrammatic view for the process of taking the consolidated patient data collected in the patient visit and processing it to provide a condensed and more focused profile of a particular patient. This profile will result in a unique Patient Identification Number (PIN). This is illustrated in a block 202. The process is illustrated at block 204. This process basically takes all the data that can be provided which is an ordered set of data and is designed to collect data primarily for the purpose of determining factors that relate to patients with chronic pain. For example, one of the first steps of screening a chronic pain patient is to collect data made during a brief psychosocial screening which asked the following questions:
Activities: how is your pain affecting your life (i.e. Sleep, appetite, physical activities, and relationships)?
Coping: how do you deal/cope with your pain (what makes it better/worse)?
Think: do you think your pain will ever get better?
Upset: have you been feeling worried (anxious)/depressed (down, blue)?
People: how do people respond when you have pain?

In dealing with the overall interview, a Standardized Pain Assessment can be performed which has been developed to evaluate patients' attitudes, beliefs, symptoms, motions, quality of life, and expectancies about themselves and the healthcare system. These, of course, can change every time a patient visits the physician's office. These are shown in the following table:

### Sample of Standardized Tools for Chronic Pain Assessment

| | | |
|---|---|---|
| Measure | Number of items | Domain assessed |

Unidimensional pain measures

| | | |
|---|---|---|
| Numerical Rating Scale | 1 | Pain intensity using a numbered scale |
| (NRS) | | (e.g. 0-10, 0-100) |
| Verbal Rating Scale (VRS) | 1 | Pain intensity using verbal descriptors |
| | | (e.g. mild, moderate, severe) |
| Visual Analog Scale (VAS) | 1 | Pain intensity using 10 or 100 mm line, |
| | | anchored by no pain and worst possible |
| | | pain |
| Facial Pain Scale (FPS) | 1 | Pain intensity using a range of facial |
| | | expressions |
| Pain thermometer | 1 | Pain intensity using a depicted |
| | | thermometer to rate pain |

Pain quality and location

| | | |
|---|---|---|
| McGill Pain Questionnaire | 20 | Pain quality, location, exacerbating, and |
| (MPQ) | | ameliorating factors |
| Short-form-McGill Pain | 22 | Pain quality, location, exacerbating, and |
| Questionnaire-2 (SF-MPQ-2) | | ameliorating factors |
| Neuropathic Pain Scale | 10 | Neuropathic pain qualities |
| (NPS) | | |
| Regional Pain Scale (RPS) | 19 Sites | Extent of body pain |

Pain interference and function: general

| | | |
|---|---|---|
| Pain Disability Index (PDI) | 7 | Pain disability and interference of pain |
| | | in functional, family, and social |
| | | domains |
| Brief Pain Inventory (BPI) | 32 | Pain intensity and interference of pain |
| | | with functional activities |
| PROMIS pain interference | Interference | Pain interference and behaviours related |
| and pain behaviours item | Bank = 41; | to the impact of pain |
| banks | Behaviours | |
| | Bank = 39 | |
| Functional Independence | 18 | Physical and cognitive ability, burden of |
| Measure | | care |

Pain interference and function: disease specific

| | | |
|---|---|---|
| Western Ontario | 24 | Pain and function in people with |
| MacMaster Osteoarthritis | | osteoarthritis |
| Index (WOMAC) | | |
| Fibromyalgia Impact | 20 | Health status for people with |
| Questionnaire (FIQ) | | fibromyalgia |
| Roland-Morris Disability | 24 | Pain and disability for people with back |
| Questionnaire (RDQ) | | pain |

HRQOL

| | | |
|---|---|---|
| Medical Outcomes Study | 36 | Mental and physical health |
| Short Form Health Survey | | |
| (SF-36) | | |
| West Haven-Yale | 60 | Pain severity, interference, mood, |
| Multidimensional Pain | | activities, sense of control, support, |
| Inventory (MPI) | | quality of life |
| EuroQOL (EQ-5D) | 5 | Health status, pain, and mood |
| Sickness Impact Profile | 136 | Physical and psychosocial dysfunction |
| (SIP) | | |

Psychosocial measures

| | | |
|---|---|---|
| Beck Depression Inventory | 21 | Depressive mood |
| (BDI) | | |
| Profile of Mood States | 65 | Mood and emotional functioning |
| (POMS) | | |
| Symptom Checklist-90 | 90 | Multiple domains of psychological |
| Revised (SCL-90R) | | functioning |
| Pain Catastrophizing Scale | 13 | Catastrophic thoughts related to pain |
| (PCS) | | |
| Coping Strategies | 10 | Coping strategies for chronic pain |
| Questionnaire (CSQ) | | |

Observational pain assessment

| | | |
|---|---|---|
| Pain Behaviour Checklist | 16 Categories | Observational measure to assess |
| (PBC) | | patient's pain behaviours |
| Real-time assessment of | 5 Categories | Real-time assessment of pain behaviours |
| pain behaviour | | integrated with a standardized |
| | | assessment |

The patient can also be asked to assess the pain intensity via a self-report measure, report the pain quality and pain location in addition to the pain intensity, the pain interference with function and quality of life, the emotional distress and coping issues that the patient may be undergoing, the overt expressions of pain, etc. All of these responses will provide valuable information to the patient profile. However, the correlation in this data is of such nature that certain tests in certain responses to questions and the such had a higher weight in the decision-making process as to the reduction of opioid use. This also greatly affects the combination of opioid use with alternative compounds, and it also, as will be described hereinbelow, will affect the determination of what compound formulation will correlate with the highest degree of opioid reduction. It may be that a patient can function with a 60% opioid reduction by substituting a particular compound formulation involving such things as topical analgesics and the such. It is the determination of this compound formulation that will be determined by the system and method set forth hereinbelow. However, once the particular tests and assessments that relate to chronic pain have been determined to be important, they can be reduced to just the raw values or two normalized values that can be placed in various bins associated with various fields in the patient PIN. This patient PIN is a Pain Centric PIN for a particular patient. There is one field that provides a unique code for the patient, a field 210, which is a Patient Information Profile (PIP). This is the basic patient profile that does not change. This will identify the patient, whereas the Patient Centric Patient PIN 202 identifies the patient profile at a particular time associated with chronic pain as experienced by the patient at that particular time. This chronic pain may vary as a result of the pain medication the patient has been taking, the mental attitude of the patient, or other external things that have changed in the patient's life since, for example, the last time that the patient had been profiled from a patient centric point of view.

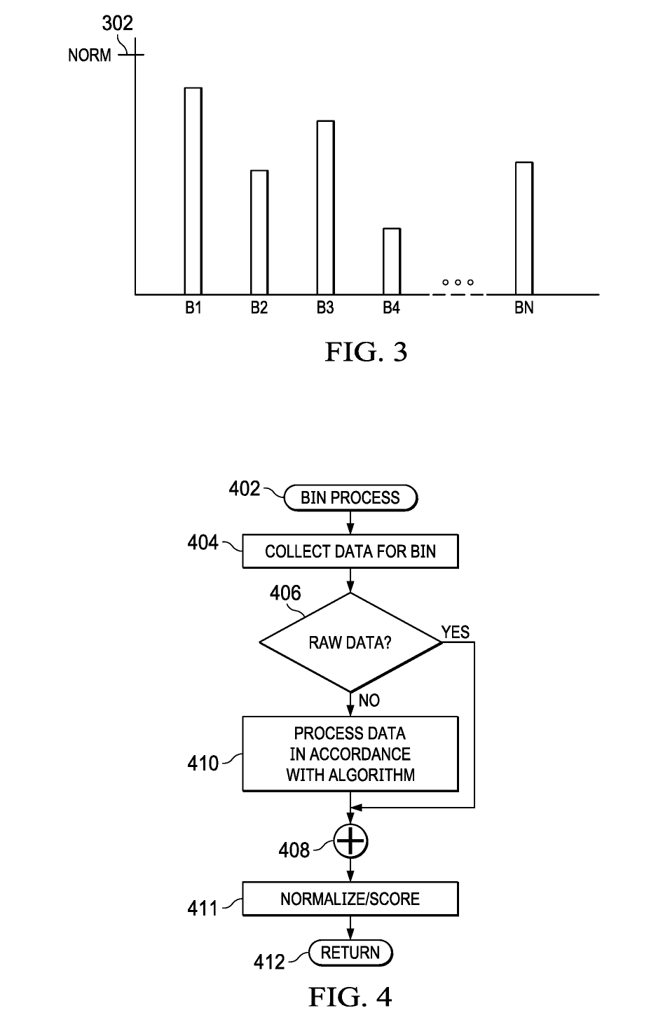

FIG. 3 illustrates a histogram illustrating how the values in the bins 206 are distributed. All of the values, in this example, are normalized to a value 302. They could, of course, be the actual values. Each of the bins will have a different value associated therewith, resulting in a unique code for that particular patient at that particular time from a pain centric point of view. This particular unique code will probably change each time the patient is evaluated. A number of the bins could actually be associated with the actual drugs or compound formulation that the patient is currently taking.

FIG. 4 illustrates a flowchart depicting the overall binning process, which is initiated at a block 402 and then proceeds to a block 404 wherein all of the data is connected for a particular bin. The program then proceeds to block 406 to determine if basically the raw data from the test or the questionnaire is to be input to the associated bin. So, the program flows to the input of a summation node 408 and, if not, the program flows along a “N” path to a function block 410 in order to process data in accordance with a predetermined algorithm or some type of consolidation process. The program then flows to a function block 410 to normalize/score a particular value. The term “score” refers to a process whereby a group of tests or answers to questions may be evaluated and given a final value of between 0 to 10, for example. It could be that all of these questions answered by the patient in the written assessment are lumped together, each given a weight and then summed and normalized to provide just an overall score for the assessment operation. This is compared to provide each and every answer as an input to a separate bin 206. The program flows to a return block 412.

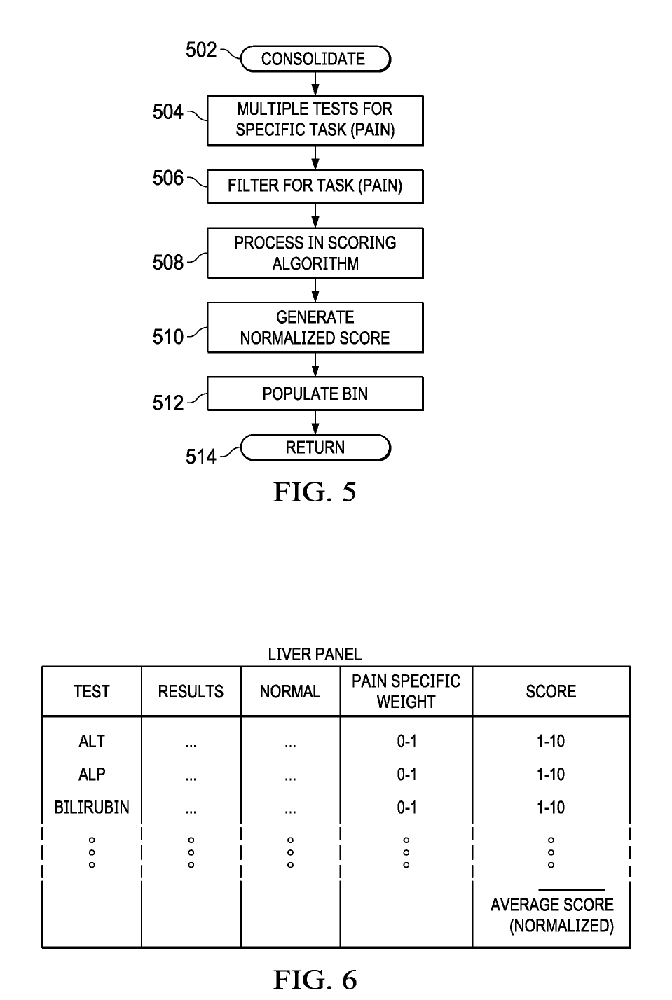

Referring now to FIG. 5, there is illustrated a flowchart depicting the overall consolidation process. This is initiated at a block 502 and then proceeds to a block 504 in order to process multiple tests for a specific pass, in this example as described hereinabove, for evaluating chronic pain in a particular patient. Again, this could be an assessment questionnaire, or it could be a lab tests such as liver test, as one example. There is then provided a filter in a process step 506 for the particular task to throw some tests out which are relatively minor as to the overall assessment of what type of compound would reduce opioid use, for example. If, for example, a liver panel were ordered, there may be certain aspects in the overall results of that test that are known to have a little correlation to that particular determination and these are filtered out. The program then flows to a process step 508 wherein, after the filtering step, the process scores the results of the tests with some particular algorithm, this being a consolidation algorithm. The process then flows to a process block 510 in order to generate a normalized score and then to a process block 512 in order to populate the associated bin and then to a return block 514.

Referring now to FIG. 6, there is illustrated a method for consolidating a liver panel, for example. In this example, there will be a plurality of test results in one column, this being the title of the test and this will provide the actual results of the tests as compared to the normal values expected for that test. In the consolidation process, each of the tests will be given a weight from 0 to 1, and then the figure value will be normalized to a value from 1 to 10, this being the score. For example, the first test, that labeled “ALT” for “Alanine Aminotransferase,” which is an enzyme mainly found in the liver which is usually considered a good test for detecting hepatitis, is defined in the first column labeled “Test” with results provided therefore and a column showing the normal ranges, which is usually age-based and then the weight with a value between 0 to 1 and then a score from 1-10. Typical contents of a liver panel are as follows:
Alanine aminotransferase (ALT)—an enzyme mainly found in the liver; the best test for detecting hepatitis
Alkaline phosphatase (ALP)—an enzyme related to the bile ducts but also produced by the bones, intestines, and during pregnancy by the placenta (afterbirth); often increased when bile ducts are blocked.
Aspartate aminotransferase (AST)—an enzyme found in the liver and a few other organs, particularly the heart and other muscles in the body
Bilirubin—two different tests of bilirubin often used together (especially if a person has jaundice): total bilirubin measures all the bilirubin in the blood; direct bilirubin measures a form that is conjugated (combined with another compound) in the liver.
Albumin—measures the main protein made by the liver; the level can be affected by liver and kidney function and by decreased production or increased loss.
Total protein (TP)—measures albumin and all other proteins in blood, including antibodies made to help fight off infections
Depending on the healthcare provider and the laboratory, other tests that may be included in a liver panel are:
Gamma-glutamyl transferase (GGT)—another enzyme found mainly in liver cells
Lactate dehydrogenase (LD)—an enzyme released with cell damage; found in cells throughout the body
Prothrombin time (PT)—the liver produces proteins involved in the clotting (coagulation) of blood; the PT measures clotting function and, if abnormal, may indicate liver damage.
Alpha-feto protein (AFP)—associated with regeneration or proliferation of liver cell
Autoimmune antibodies (e.g., ANA, SMA, anti-LKM-1)—associated with autoimmune hepatitis

When treating patients with opioid dependence, only certain tests resulting from the liver panel will be relevant or will be important to chronic pain. For example, patients receiving certain drugs such as, for example, buprenorphine, may have some adverse events associated with increases in serum aminotransferase levels. These may actually be the result of an individual with Hepatitis C. By understanding the comorbidity in such a situation, it is important to assign a weight the ALT and ALS test results. Another enzyme that is critical for the metabolism of some opioids is cytochrome P450, wherein a number of opioids are affected by this particular enzyme, such as codeine, hydrocodone, oxycodone, tramadol, fentanyl, and methadone. Again, this table of FIG. 6 is by way of example of any test that can be performed and importance of that particular test or group of tests that may have some importance to a chronic pain patient. There may be other portions of the liver panel, for example, that are more important to heart disease, such as lipid levels. These, of course, would be given little or no weight. A table for all of tests associated with the liver tests is as follows:

| Type of liver | | | | | |
|---|---|---|---|---|---|
| condition or | | | | | |
| disease | Bilirubin | ALT and AST | ALP | Albumin | PT |
| Acute liver | Normal or | Usually greatly | Normal or | Normal | Usually |
| damage (due, | increased | increased (>10 | only | | normal |
| for example, to | usually after | times); ALT is | moderately | | |
| infection, | ALT and AST | usually higher | increased | | |
| toxins or | are already | than AST | | | |
| drugs, etc.) | increased | | | | |
| Chronic forms | Normal or | Mildly or | Normal to | Normal | Normal |
| of various liver | increased | moderately | slightly | | |
| disorders | | increased; ALT | increased | | |
| | | is persistently | | | |
| | | increased | | | |
| Alcoholic | Normal or | AST is | Normal or | Normal | Normal |
| Hepatitis | increased | moderately | moderately | | |
| | | increased, | increased | | |
| | | usually at least | | | |
| | | twice the level | | | |
| | | of ALT | | | |
| Cirrhosis | May be | AST is usually | Normal or | Normal or | Usually |
| | increased but | higher than ALT | increased | decreased | prolonged |
| | this usually | but levels are | | | |
| | occurs later in | usually lower | | | |
| | the disease | than in alcoholic | | | |
| | | disease | | | |
| Bile duct | Normal or | Normal to | Increased; | Usually | Usually |
| obstruction, | increased; | moderately | often greater | normal but | normal |
| cholestasis | increased in | increased | than 4 times | if the | |
| | complete | | what is normal | disease is | |
| | obstruction | | | chronic, | |
| | | | | levels may | |
| | | | | decrease | |
| Cancer that has | Usually normal | Normal or | Usually | Normal | Normal |
| spread to the | | slightly | greatly | | |
| liver | | increased | increased | | |
| (metastasized) | | | | | |
| Cancer | May be | AST higher than | Normal or | Normal or | Usually |
| originating in | increased, | ALT but levels | increased | decreased | prolonged |
| the liver | especially if | lower than that | | | |
| (hepatocellular | the disease has | seen in alcoholic | | | |
| carcinoma, | progressed | disease | | | |
| HCC) | | | | | |
| Autoimmune | Normal or | Moderately | Normal or | Usually | Normal |
| | increased | increased; ALT | slightly | decreased | |
| | | usually higher | increased | | |
| | | than AST | | | |

Note that only conditions that will be associated with a chronic pain patient and the reduction opioid dependency would be of interest.

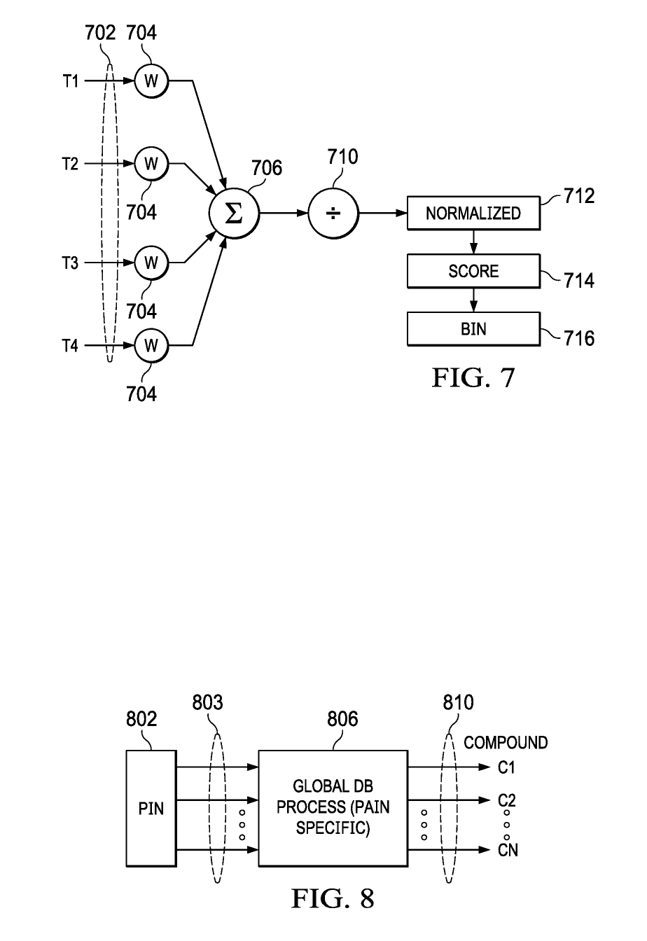

Referring now to FIG. 7, there is illustrated a diagrammatic view of how to consolidate all of these tests into a single number, as it may be that the necessary value to provide is a single score for a common test of, for example, a liver panel. In this example in FIG. 7, there are provided a plurality of inputs 702 that each represent the results of a particular test. They are each processed through a particular weight value in a block 704 and then results summed together in a summing junction 706. The output is then divided by the number of tests in a block 710 and normalized in a block 712. This will provide a normalized value for the results, which can then be translated to a score from 1-10 in a block 714. This is a value that is stored in the bin, as indicated by block 716. Thus, all or a certain portion of the tests can be summed together and normalized, with the resulting score representing a portion of the PIN for the particular patient at the time that they are evaluated. It is again important to note that, each time a patient is evaluated, the results may be different. This is a function of the drugs that have been prescribed and the progression of their particular opioid dependence. For example, between two visits to a physician, the therapy prescribed by the physician may have reduced the opioid dependence by fifty percent. This would be ascertained through a questionnaire and that would be one input to the patient's PIN. This combined with the actual drugs being received, which is also part of the patient's PIN, would be provided as input to the database and comparing this particular patient's PIN with the results in the database, this being a global database. It is noted that one would expect a different result to be projected for the suggested therapy for that patient at that time. This is due to the fact that the first time the patient was evaluated and placed in the database, the suggestion might be to change the drug therapy. If the drug therapy has worked, the second time the information is placed into the database for comparison with the global database, a different result would come back.

Referring now to FIG. 8, there is illustrated a block diagram of a global database that is a pain specific global database, i.e., the data provided thereto is specifically for the purpose of creating a model that will receive information from a particular patient, i.e., through that patient's particular PIN at the time of their evaluation, process it through the model based upon a large amount of data from other patients, and provide some type of suggested output. Here, the patient's PIN is provided in a block 802, and all of the inputs comprise an input vector lines 803 to the local database 806. This provides a resultant vector on output 810. In this particular example, the resultant vector is the actual compound that would be suggested for a particular patient.

This particular output, that of the compound, is just one example of what the result could be. The particular compound could be a combination of multiple constituents that had been determined through an observational survey study which looked at patients over a certain age range having chronic musculoskeletal and neuropathic pain. As an example, a topical drug with the following compounding could be one form of a compound:
Flurbiprofen (20%)—anti-inflammatory
Amitrityline (5%)—Antidepressant
Magnesium Chloride (10%)—Salt
Gabapentine (6%)—Anti Seizure
Bupivicaine (2%)—Local Antiesthetic
Other transdermal gel

This particular compound combines an anti-inflammatory, antidepressant, a salt, an anti-seizure medicine, and a local anesthetic in a transdermal gel base. This provides the patient with a topical drug compound that can be used to reduce opioid dependence. Through the observational study, patients with a particular profile, i.e., a unique PIN at the time of the study, are evaluated at a later time to determine the results. The first result, of course, is the percentage of opioid reduction, and the second may be the actual percentage by weight of the compounds. The particular percentages noted hereinabove are percentages by weight which are determinable by the observational study as a normal value. It may be that the clinician selecting the original percentage values selected those based on known therapeutic results at a particular dosage. Also, price may be a factor.

Once a therapeutic level of a particular drug is determined to provide the therapeutic result of acceptable opioid reduction, and this can be done through trial and error via variation of the percentages, it is possible to vary those percentages based upon price. One formula for doing this is to vary the particular percentage weight of a particular compound from a minimum percentage weight to a maximum percentage weight. One formula for that is to take the norm, as determined through the observational study, and reduce it to 25% of the dosage on one end of the price perspective and multiplied by factor of two to determine the maximum dosage from a price perspective. This price can be one factor for determining the percentage weight of a particular compound. Additionally, substitutes for any of the drugs could be provided by utilizing generics or the such.

Thus, by utilizing a global database which has information stored therein that correlates particular information associated with the information from a PIN with a desired or predicted result, any PIN from a patient can be input to the global database and mapped through that database to provide a prediction. For example, the prediction may be that a particular PIN for a particular patient has been put in, and a particular compound has been put in, and this information then “mapped” through global database process to provide an estimate of, or a prediction of, a potential reduction in opioid dependency. Alternatively, the information from a PIN of the patient could be input to the process in addition to a target range of opioid reduction and a suggestion or prediction made as to what compound, a topical drug compound for example, would be suggested. Since the model which the input information is mapped is based on a larger database of results, this will allow mapping based on a relatively nonlinear system.

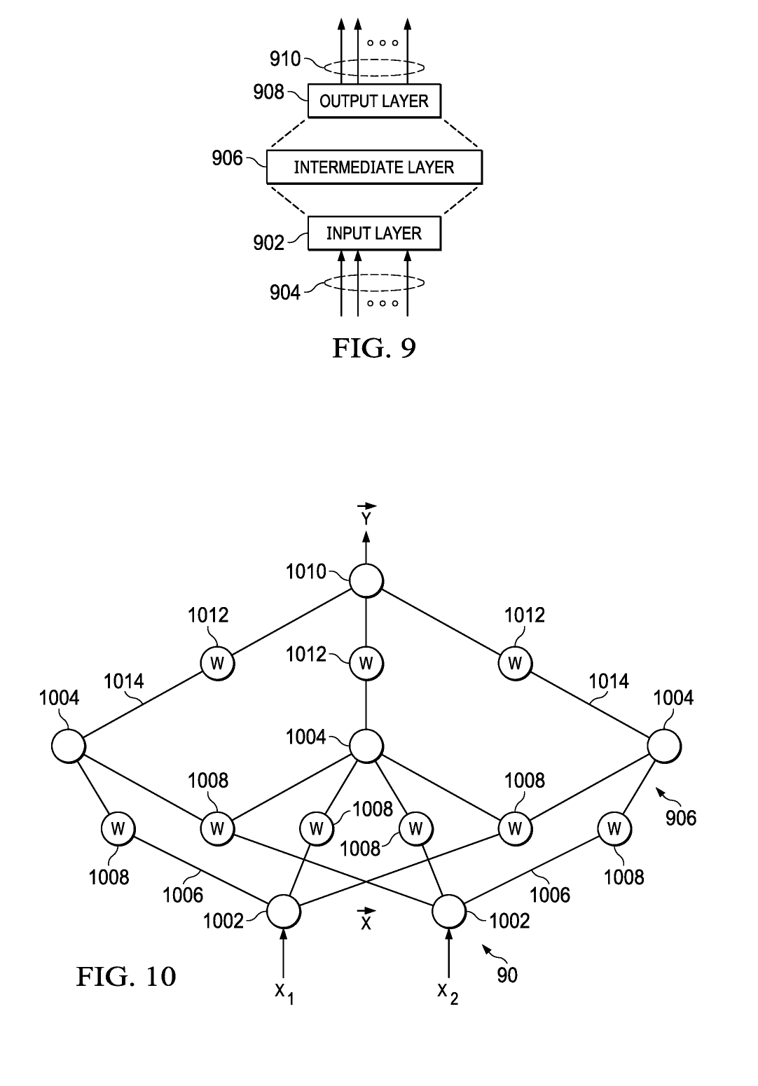

Referring now to FIG. 9, there is illustrated a diagrammatic view of one example of a model through which input data can be mapped to provide an estimate or a prediction on the output thereof. This is a neural network, which is a non-linear network. These type of networks can provide predictive results based on nonlinear system, wherein the human body and the overall evaluation thereof is a fairly nonlinear system. The neural network is comprised of an input layer 902 that receives an input vector 904 comprised of a plurality of input values, these being the values from the PIN. The input layer 902 is interconnected to one side of an intermediate layer 906, which is interconnected to an output layer 908. The output layer 908 is comprised of a vector 910 of a plurality of predicted outputs. The intermediate layer 906 and the interconnections thereto, once the interconnections are made, represent a model of the overall system, this model been trained upon the collected historical data.

Referring now to FIG. 10, there is illustrated a more detailed diagram of a sample neural network. The input layer 902 is represented by two input nodes 1002 associated with a vector \vec{x} comprised of two inputs. There are provided in the intermediate layer 906 three nodes 1004 to which each of the nodes 1002 is mapped. Thus, there will be three interconnections between each of the nodes 1002 and each of the nodes 1004. Each of these interconnections is defined by interconnection line 1006. Each of these interconnects has associated therewith a weight 1008. Thus, the input vector \vec{x} is comprised of two inputs x₁ and x₂ which each are interconnected to each of the nodes 1004. If weight is defined as ω, then the formula for the input to each of the nodes 1004 for the first input vector x₁ will be: ωx₁. Each of the nodes 1004 in the intermediate layer 906 has associated therewith some type of function which is basically an activation function which “fires” this node to generate an output is typically a sigmoid function. Each of the nodes 1004 is individually mapped to a single output node 1010 that outputs an output the vector \vec{y}, it being noted that multiple output nodes 1010 could be provided with each of the nodes 1004 mapped to or interconnected to each of the nodes 1010 in a multiple note output. Each of these nodes 1004 is interconnected to the respective output node 1010 through a respective weight 1012 and a respective interconnect 1014. These weights are learned through such techniques as back propagation. In back propagation, a set of data is provided wherein a known output for a set of data values for the input vector \vec{x} is input to the network with an error determined between the mapping of this set of input data for that input vector through the intermediate layer 906 to the output. The weights are iteratively adjusted until the error is minimized. It is necessary to iteratively go through an entire set of data multiple times in order to reduce the error. This will result in a trained the model of the system represented by the database.

As an example, consider the situation wherein the desire is merely to determine for a given patient with a given PIN what their opioid reduction would be for a given compound. The PIN is input to the model, as well as the compound constituents and the percentages. The system will process this and output a predicted opioid reduction for that individual. Of course that means that the input vector upon which the model was trained was comprised of the elements of the PIN of patients in addition to the corresponding percentages of the compound. What that means is that the original database must have incorporated therein all the information from the patient in addition to the constituents associated with the compound at those percentages and some value of the opioid reduction determined therefrom. Thus, a patient would have a first PIN generated before taking a particular compound with a particular set of constituents at a particular defined percentage weight for each constituent and put the initial data from their initial PIN into the database in addition to the exact constituent distribution of the topical drug that they utilized and opioid reduction achieved after the use thereof. There, of course, would be required a large data in order to cover all possible combinations of patients and the different percentages by weight of the constituents in a particular compound. This is just one example.

In another example, the model can be trained to actually predict a compound, the constituents associated therewith and percentages by weight of the constituents contained therein. This would require, for a given set of data for a given input vector to be comprised of the patient PIN at the initial point in a study, a given opioid reduction for that patient after completion of the study, and a configured compound that was provided to the patient. Thereafter, all that is required is to put in the PIN for the new patient in addition to inputting therein a desired opioid reduction value or range of values as part of the input vector. Since the network is trained on that particular set of input vectors and that particular set of output vectors, a prediction can be made as to the percentage by weight of the constituents. There might, in fact, be required a separate model for each different compound such that the patient PIN can be processed through different compounds. In addition, once this particular patient with their initial PIN has been processed to the system and a prediction made as to what particular compound should be utilized, a later PIN from that patient and results can be input to the model for training there on.

Although the preferred embodiment has been described in detail, it should be understood that various changes, substitutions and alterations can be made therein without departing from the spirit and scope of the invention as defined by the appended claims.

1. An opiate reduction treatment system, the system comprising:
a PIN generator for creating a Patient Identification Number (PIN) unique to a given patient, wherein the PIN includes one or more fields, and wherein the one or more fields each include a scored value converted from raw data corresponding to one or more test results, each scored value associated with a defined portion of a health profile of the given patient;
a database including test results for a plurality of PINs for a plurality of patients and associated compound constituents provided to each of the plurality of patients, and known treatments; and
a neural network, including:
an input layer configured to receive an output of a PIN for a given patient from the PIN generator and compound constituents as input values,
an output layer configured to provide an opioid reduction treatment prediction, and
an intermediate layer configured to store a representation of the database, and map the input layer to the output layer through the stored representation.

2. The system of claim 1, wherein the scored value is created from one or more inputs from the raw data that are weighted according to associated test types and normalized.

3. The system of claim 1, wherein the one or more fields of the PIN includes a code assigned to a patient.

4. The system of claim 3, wherein the code assigned to the patient is a Patient Information Profile (PIP), wherein the PIP identifies the patient.

5. The system of claim 1, wherein at least one of the one or more fields of the PIN corresponds to a particular test.

6. The system of claim 1, wherein at least one of the one or more fields of the PIN corresponds to a compound formulation.

7. The system of claim 1, wherein the scored value is a value within a number range.

8. The system of claim 7, wherein the number range is a range between 1 and 10.

9. The system of claim 1, wherein the PIN represents a patient pain profile at a first point in time.

10. The system of claim 9, wherein the neural network is further configured to:
receive an output of another PIN representing a patient pain profile at a second point in time;
predict another opioid reduction treatment using the other PIN; and
store a revised treatment plan in the database.

11. A method for providing an opiate reduction treatment, comprising:
generating a Patient Identification Number (PIN) including one or more fields;
collecting raw data corresponding to one or more test results;
converting the raw data into a scored value;
storing the scored value in one of the one or more fields of the PIN;
predicting an opioid reduction treatment for a patient, including
providing as input values an output of the PIN and compound constituents to an input layer of a neural network,
applying, by an intermediate layer of the neural network, the input values and compound constituents information to a stored representation of a database, wherein the database includes test results for a plurality of PINs for a plurality of patients and associated compound constituents provided to each of the plurality of patients, and
generating, by an output layer of the neural network, an opioid reduction treatment prediction; and
delivering to a patient an opioid reduction treatment corresponding to the opioid reduction treatment prediction.

12. The method of claim 11, wherein converting the raw data into the scored value includes:
creating one or more inputs from the raw data;
applying a weight to the one or more inputs to generate one or more weighted results, each one of the one or more weighted results corresponding to one of the one or more inputs;
summing the one or more weighted results to generate a summed output;
dividing the summed output by a number of tests to generate a result; and
translating the result into the scored value.

13. The method of claim 11, wherein the one or more fields of the PIN includes a code assigned to a patient.

14. The method of claim 13, wherein the code assigned to the patient is a Patient Information Profile (PIP), wherein the PIP identifies the patient.

15. The method of claim 11, wherein at least one of the one or more fields of the PIN corresponds to a particular test.

16. The method of claim 11, wherein at least one of the one or more fields of the PIN corresponds to a compound formulation.

17. The method of claim 11, wherein the scored value is a value within a number range.

18. The method of claim 17, wherein the number range is a range between 1 and 10.

19. The method of claim 11, wherein the PIN represents a patient pain profile at a first point in time.

20. The method of claim 19, further comprising:
providing an output of another PIN representing a patient pain profile at a second point in time;
predicting another opioid reduction treatment using the other PIN; and
storing a revised treatment plan in the database.
